(19) United States
(12) Patent Application Publication
(10) Pub. No.: US 2012/0045816 A1
(43) Pub. Date: Feb. 23, 2012

(54) Novel Glycosylated Polypeptides

(75) Inventors: Darius Ghaderi, San Diego, CA (US); Ajit Varki, La Jolla, CA (US)

(73) Assignee: Sialix, Inc.

(21) Appl. No.: 13/183,385

(22) Filed: Jul. 14, 2011

Related U.S. Application Data
(63) Continuation-in-part of application No. 13/062,069, filed on Apr. 18, 2011.
(60) Provisional application No. 61/365,727, filed on Jul. 19, 2010.
(60) Provisional application No. 61/095,414, filed on Sep. 9, 2008.

(51) Int. Cl. C12N 9/06; C07K 14/575; C07K 16/00; C07K 14/52; C07K 14/535; C07K 14/585; C07K 14/49; C07K 14/475; C07K 14/51; C07K 14/565; C07K 14/57; C07K 14/56; C07K 14/555; C12N 9/48; C12N 9/24; C12N 9/26; C12N 9/16; C12N 9/22; C07K 16/46

(52) U.S. Cl. 435/191; 530/387.3; 530/395; 530/397; 530/388.1; 530/351; 530/307; 435/212; 435/200; 435/201; 435/196; 435/199

(57) ABSTRACT

Provided herein are glycosylated polypeptide compositions with substantially reduced Neu5Gc content. The glycosylated polypeptides compositions with substantially reduced Neu5Gc content can be obtained from cell sources cultured with Neu5Gc competitor or from non-human animal sources fed a diet supplemented with Neu5Gc competitor. Also provided herein are methods of treating a human subject with said compositions.

### CROSS-REFERENCE TO RELATED PATENT APPLICATIONS

This application claims the benefit of U.S. Provisional Application Ser. No. 61/365,727 filed Jul. 19, 2010, entitled “Novel Glycosylated Polypeptides.” This application is a continuation-in-part of U.S. patent application Ser. No. 13/062,069, filed Apr. 18, 2011, entitled “Elimination of a Contamination Non-Human Sialic Acid by Metabolic Competition” which claims benefit of PCT Application Serial No. PCT/US2009/056361, filed Sep. 9, 2009, which claims benefit of U.S. Provisional Application Ser. No. 61/095,414 filed Sep. 9, 2008, which are all incorporated herein by reference in their entirety and for all purposes.

### FIELD OF THE INVENTION

This invention relates to glycosylated polypeptide compositions and their uses. The glycosylated polypeptides of the invention include, for example, polypeptides with reduced Neu5Gc.

### BACKGROUND OF THE INVENTION

Glycosylated polypeptides (antibodies, growth factors, cytokines, hormones and clotting factors etc.) are often produced in mammalian expression systems. Non-human cell lines, such as rodent cell lines, used to produce glycosylated polypeptides generate N-glycans similar to those of humans, with a few exceptions. For example, the CMP-N-acetylneuraminic acid hydroxylase (CMAH) gene responsible for CMP-Neu5Gc production from CMP-N-acetylneuraminic acid (CMP-Neu5Ac) is irreversibly mutated in all humans¹, but intact in non-human mammalian cells often used to produce glycosylated polypeptides. Thus, polypeptides produced from human cells generally lack Neu5Gc. However, Neu5Gc can be taken up from animal products present in the culture medium and then metabolically incorporated into secreted glycoproteins by human cells². Thus, even human cells cultured with animal-derived supplements will secrete glycoproteins bearing Neu5Gc. Notwithstanding the capability of human cells to incorporate Neu5Gc into glycoproteins, Neu5Gc is not part of the normal glycosylation pattern of human proteins and humans have been observed to generate high levels of circulating anti-Neu5Gc antibodies to the Neu5Gc-contaminated glycoproteins. Repeated injections of Neu5Gc-carrying agents may load human tissues with this non-human sugar which, in combination with anti-Neu5Gc antibodies, mediate chronic inflammation and potentially facilitate progression of diseases such as cancer³ and atherosclerosis⁴. Thus, chronic use of Neu5Gc-bearing therapeutics may increase the risk of developing such diseases. Furthermore, Neu5Gc-bearing therapeutics can be cleared faster from the bloodstream due to anti-Neu5Gc antibodies and therefore have lowered efficacy relative to the same therapeutic with a lowered Neu5Gc content.

### SUMMARY OF THE INVENTION

The present invention is based on methods for the production of glycosylated polypeptides, including polypeptides with substantially reduced or no Neu5Gc content. Compositions of the invention include recombinant human glycosylated polypeptides or non-human animal glycosylated polypeptides in which Neu5Gc is substantially reduced or eliminated.

In one embodiment, the compositions include, but are not limited to, a polypeptide such as a monoclonal antibody, Fc-fusion protein, hormone, cytokine, clotting factor, enzyme inhibitor, enzyme and antiserum.

In an embodiment, the monoclonal antibody is Tocilizumab, Bevacizumab, Alemtuzumab, Trastuzumab, Adalimumab, Rituximab, Golimumab, Ustekinumab, Panitumumab, Omalizumab, Ibritumomab tiuxetan, Tositumomab-I131, Eculizumab, Canakinumab, Gemtuzumab ozogamicin, Ofatumumab, Palivizumab, Natalizumab, Cetuximab, Infliximab, Abciximab, Basiliximab, Daclizumab, Certolizumab pegol, or Ranibizumab.

In an embodiment, the Fc-fusion protein is Alefacept, Rilonacept, Etanercept, Abatacept, or Romiplostim.

In an embodiment, the hormone is Follitropin beta, Follitropin alfa, Luteinizing hormone, Osteogenic Protein-1 (BMP-7), Choriogonadotropin alpha, Thyrotropin alfa, Somatropin, keratinocyte growth factor, Calcitonin, or Platelet-derived growth factor (PDGF).

In an embodiment, the cytokine is Darbepoetin alfa, Interferon beta-1a, Epoetin beta, Epoetin alfa, Interferon beta-1a, Interferon gamma-1b, Interferon alfacon-1, Interferon alfa-2b, interleukin-1 receptor antagonist (IL-1Ra), Pegfilgrastim, Des-Pro Interleukin-11, G-CSF, IL-2/diphtheria toxin fusion protein, Peginterferon alfa-2a, Aldesleukin (IL-2), or Interferon alfa-2a.

In an embodiment, the clotting factor is Coagulation factor VIII, Coagulation Factor VIIa, Antihemophilic factor, Coagulation Factor IX, Antihemophilic Factor, or Drotrecogin alfa (Activated Protein C).

In an embodiment, the enzyme is Alteplase, Laronidase, Imiglucerase, agalsidase-O, yaluronidase, Alglucosidase alfa, N-acetylgalactosamine 4-sulfatase, Human DNase, Tenecteplase, Idursulfase, Collagenase, or Rasburicase.

In one embodiment, the glycosylated polypeptide is recombinant (i.e., produced by recombinant protein expression methods). In other embodiments the glycosylated polypeptide is mammalian, including human and/or non-human polypeptides.

In an embodiment, the composition (e.g., a glycosylated polypeptide) is derived from a cell line fed Neu5Ac, e.g. cultured in media containing Neu5Ac. In an embodiment the cell line is derived from a non-human animal. In another embodiment the cell line is derived from a human. In certain embodiments the culture cells are hybridomas, Chinese Hamster Ovary (CHO) cells, murine myeloma cells, murine C127 cells, Baby Hamster Kidney (BHK) cells, HT-1080 or Human embryonic kidney cells (HEK293).

“Polypeptide” refers to a polymer in which the monomers are amino acids and are joined together through amide bonds, alternatively referred to as a polypeptide. Additionally, unnatural amino acids, for example, β-alanine, phenylglycine and homoarginine are also included. As used herein, “polypeptide” refers to both glycosylated and unglycosylated polypeptides. Also included are polypeptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The term polypeptide includes molecules that are commonly referred to as peptides or proteins.

An “isolated” or “purified” glycosylated polypeptide or biologically-active portion thereof is substantially free of cellular material or other contaminating polypeptides from the cell or tissue source from which the glycosylated polypeptide is derived.

As used herein, “glycosylated polypeptide” means a polypeptide having at least one carbohydrate moiety covalently linked thereto. It is understood that a glycosylated polypeptide may be a “therapeutic glycosylated polypeptide.” The term “glycosylated polypeptide” can be used interchangeably herein with the terms “glycopolypeptide,” “glycopeptide” and “glycoprotein.”

As used herein, “recombinant” means derived from genetic engineering, e.g. a recombinant polypeptide isolated from a cell or organism wherein the nucleic acid coding for the polypeptide is from another organism, such a human gene coding for a human protein that is expressed in a Chinese Hamster Ovary (CHO) cell.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, when required for proper expression and function.

Compositions of the invention do not include human glycosylated polypeptides obtained directly from humans wherein the human glycosylated polypeptides substantially lack Neu5Gc, e.g. human glycosylated polypeptides obtained from human blood or tissue.

As used herein “Neu5Gc” means N-glycolylneuraminic acid, which is converted from the sialic acid N-acetylneuraminic acid (Neu5Ac) by the activity of cytidine monophosphate hydroxylase (CMAH).

As used herein a “Neu5Gc competitor” is a compound, such as a sugar, that can compete with Neu5Gc in cell culture or non-human animal such that an alternate sugar can replace Neu5Gc on glycosylated polypeptides.

As used herein, “substantially reduced” in Neu5Gc means that the human glycosylated polypeptide has a lower mol fraction of Neu5Gc than what is obtained when the human glycosylated polypeptide is produced in a non-human animal source, including, but not limited to (1) in a non-human animal cell line or (2) in a non-human animal cell line or a human cell line wherein the cell line is cultured in media that introduces Neu5Gc (such as by supplementing the media with fetal calf serum). In certain embodiments, the glycosylated polypeptides can have a mol fraction of Neu5Gc of less than 2, of less than 1, of less than 0.5, of less than 0.2, of less than 0.1, of less than 0.05, of less than 0.02, of less than 0.01, of less than 0.005, of less than 0.002, or of less than 0.001. In certain embodiments, the glycosylated polypeptide with substantially reduced Neu5Gc has a mol fraction of Neu5Gc of more than 0.01, more than 0.02, more than 0.05, more than 0.1, more than 0.5.

Neu5Gc content of a glycosylated polypeptide can be readily determined by those skilled in the art using immunodetection methods known in the art.⁵

### BRIEF DESCRIPTION OF THE FIGURES

FIG. 1: ELISA and Western-Blot Detection of Neu5Gc on Cet and Pan. Sialidase-treated and heat-treated sialidase-treated Cetuximab (Cet) and Panitumumab (Pan) evaluated for presence of Neu5Gc by ELISA (Panel A) or Western Blot (Panel B). ELISA analysis of periodate-treated Cet and Pan (Panel C). ELISA analysis of anti-Neu5Gc IgG-treated Cet and Pan (Panel D). Western Blot of anti-Neu5Gc IgG-treated Cet and Pan (Panel E). Detection of immune complex formation with Cet or Pan in whole human serum (Panel F).

FIG. 2: Effects of anti-Neu5Gc antibodies on the kinetics of Cet and Pan in mice with a human-like Neu5Gc-deficiency. ELISA analysis of therapeutic antibody clearance kinetics in Cmah null mice injected with Cet and Pan (Panel A). ELISA analysis of Neu5Gc specific antibodies in Cmah null mice were injected i.v. with Cet or Pan (Panel B). ELISA analysis of direct binding of anti-Neu5Gc antibodies to Cet and Pan (Panel C).

FIG. 3: Reduction of Neu5Gc Contamination in Glycosylated Polypeptides of Human 294T Cells and CHO Cells.Neu5Gc and Neu5Ac content, relative to total sialic acid content, of ethanol soluble (Panel A) and ethanol precipitable proteins (Panel B) analyzed by HPLC in human 293T cells grown in the presence of 5 mM Neu5Gc for 3 days. Neu5Gc content, relative to total sialic acid content, of glycoproteins in CHO cells grown in the absence or presence of 5 mM Neu5Ac in secreted proteins (Panel C) and membrane-bound proteins (Panel D) analyzed by HPLC. Western blot of proteins isolated from CHO cells grown in the absence or presence of 5 mM Neu5Ac and probed polyclonal affinity purified chicken anti-Neu5Gc antibody.

### DETAILED DESCRIPTION OF THE INVENTION

Provided herein are purified human glycosylated polypeptides with substantially reduced non-human sialic acid N-glycolylneuraminic acid (Neu5Gc) and methods for producing the same. Suitable methods for substantially reducing Neu5Gc content in human glycosylated polypeptides isolated from tissue culture or non-human animals are disclosed in U.S. Provisional Patent Application No. 61/095,414, titled “Elimination of a Contaminating Non-human Sialic Acid by Metabolic Competition,” the entire contents of which including figures are incorporated by reference herein.

In an embodiment, the human glycosylated polypeptides in which Neu5Gc is substantially reduced disclosed herein are produced by methods including the introduction of a Neu5Gc competitor to cells. For example, a Neu5Gc competitor can be used for metabolically competing out Neu5Gc, either as it enters the cells for the first time and/or when it recycles from breakdown of preexisting cellular molecules. Such Neu5Gc competitors include, but are not limited to, N-acetylneuraminic acid (Neu5Ac), available from e.g. Sigma Aldrich (Munich, Germany), E.M.D. GmbH (Berlin, Germany), MP BioMedicals (Cleveland, Ohio), Acros Organics (Geel, Belgium) and Nacalai Tesque (Kyoto, Japan), and its metabolic precursor N-acetylmannosamine (ManNAc), available from e.g. Cayman Chemical (Ann Arbor, Mich.), Santa Cruz Biotechnology, Inc. (Santa Cruz, Calif.), and Sigma Aldrich (Munich, Germany). Methods for culturing cells are well known in the art. The human glycosylated polypeptides in which Neu5Gc is substantially reduced disclosed herein are produced in human or non-human animal cells, by flooding the system with a large bolus or increasing concentrations of Neu5Gc competitor. For example, methods for producing human glycosylated polypeptides in which Neu5Gc is substantially reduced include, but are not limited to, culturing a cell line with increased amounts of N-acetylneuraminic acid (Neu5Ac) or its precursor N-acetylmannosamine (ManNAc), such as by adding such sugars as a medium supplement, for a sufficient period of time to substantially reduce Neu5Gc on glycosylated polypeptides present in a cell or cell line or product produced by a cell or cell line. Cells in culture can be supplemented with Neu5Gc competitor with at a concentration of at least 100 μM, at least 200 μM, at least 500 μM, at least 1 mM, at least 2 mM, at least 5 mM, at least 10 mM, at least 20 mM, at least 50 mM, at least 100 mM, for at least 1 day, at least 2 days, at least 3 days, at least 4 days, at least one week, at least two weeks, at least a month, or at least 3 months.

The cultured cell line from which human glycosylated polypeptides in which Neu5Gc is substantially reduced include human and non-human cells including, but not limited to, hybridomas, Chinese Hamster Ovary (CHO) cells, murine myeloma cells, murine C127 cells, Baby Hamster Kidney (BHK) cells, HT-1080 and Human embryonic kidney cells (HEK293).

Human glycosylated polypeptides in which Neu5Gc is substantially reduced can also be produced in a non-human animal, including transgenic animals engineered to express a human gene, by supplementing their feed with a Neu5Gc competitor that can effectively compete with Neu5Gc, such as N-acetylneuraminic acid (Neu5Ac) or its precursor N-acetylmannosamine (ManNAc). In an embodiment, the non-human animal is CMAH-defective. Methods for generating CMAH-defective non-human animals are disclosed in US Patent Pub. No. 2008/0166805 (U.S. Ser. No. 11/449,167). In an embodiment, the CMAH defect is “leaky” in that residual Neu5Gc contamination of the glycopolypeptides obtained from the non-human animal is still observed. This can occur, for example, where the non-human animal has residual activity of CMAH or an alternate metabolic pathway that allows the conversion of Neu5Ac to Neu5Gc. Feeding of Neu5Gc competitor to a non-human animal can be by a large bolus or by gradual increasing concentrations.

Non-human animals from which can be isolated human glycosylated polypeptides in which Neu5Gc is substantially reduced include, but are not limited to, pigs, sheep, goats and cows. Non-human glycosylated polypeptides in which Neu5Gc is substantially reduced can also be isolated from CMAH-defective non-human animals fed a diet that includes Neu5Gc competitor. For example, Pancreaze®, for which Neu5Gc is substantially reduced, can be isolated from pig fed a diet that includes Neu5Ac or ManNAc. The skilled artisan can determine the amount of Neu5Gc competitor to add to the pig feed in order to achieve substantially reduced Neu5Gc in the product isolated from the pig by the methods presented herein. Methods for correlating appropriate dosages between species are well known in the art. The human glycosylated polypeptides in which Neu5Gc is substantially reduced can be isolated from tissue or from various secretions of the non-human animal, such as milk.

Glycosylated polypeptides of the invention can have a mol fraction of Neu5Gc of less than 2, less than 1, less than 0.5, less than 0.2, less than 0.1, less than 0.05, less than 0.02, less than 0.01, less than 0.005, less than 0.002, or less than 0.001. In another embodiment, the glycosylated polypeptides of the invention can have a mol fraction of Neu5Gc that is substantially reduced but more than 0.01, more than 0.02, more than 0.05, more than 0.1, or more than 0.5 mol fraction of Neu5Gc.

While not wishing to be held by theory, the following ascending order of relative Neu5Gc content, based on cell-type or source, is expected: HEK293<CHO<BHK<Animal sources<Myeloma/Hybridoma. The absolute Neu5Gc content will also depend on the extent of glycosylation and sialylation of a given glycosylated polypeptide, which can be determined by methods disclosed herein and known in the art. For example, the Neu5Gc content of the glycosylated polypeptide can be determined in the presence and absence of Neu5Gc competitor supplement to determine how much of a reduction of mol fraction of Neu5Gc can be attained. Such methods include, but are not limited to, Western blot, ELISA and immunoprecipitation using anti-Neu5Gc antibodies or HPLC determination.

Glycosylated polypeptides substantially reduced in Neu5Gc can include, but are not limited to, monoclonal antibodies, Fc-fusion proteins, hormones, cytokines, clotting factors, enzyme inhibitors, enzymes and antisera. Tables 1 and 2, below, provide a non-exhaustive list of various commercially available recombinant therapeutic agents that when produced by the methods described herein, result in a recombinant therapeutic agent with a human-like glycosylation pattern that is substantially reduced in non-human sialic acid N-glycolylneuraminic acid (Neu5Gc). The agents are sorted generally by function in Table 1 and by cell source.

TABLE 1
FDA-APPROVED GLYCOSYLATED POLYPEPTIDES AND
CELL SOURCES FOR PRODUCTION
SORTED BY FUNCTION

| | | |
|---|---|---|
| Agent | Vendor | Cell Source |

Monoclonal Antibodies

| | | |
|---|---|---|
| Actemra ® | Genentech Inc. | CHO cells |
| Tocilizumab | | |
| Avastin ® | Genentech Inc., Hoffmann-La | CHO cells |
| Bevacizumab | Roche Ltd. | |
| Campath ® (US), | Genzyme Corp. | CHO cells |
| Mabcampath ® | | |
| (EU) | | |
| Alemtuzumab | | |
| Herceptin ® | F. Hoffmann-La Roche Ltd, | CHO cells |
| Trastuzumab | Genentech Inc. | |
| Humira ® | Abbott Laboratories | CHO cells |
| Adalimumab | | |
| Rituxan ® | Genentech | CHO cells |
| Rituximab | | |
| Simponi ® | Centocor Ortho Biotech Inc., | CHO cells |
| Golimumab | Johnson & Johnson Co., | |
| | Schering-Plough Corp. | |
| Stelara ™ | Centocor Ortho Biotech Inc. | CHO cells |
| Ustekinumab | | |
| Vectibix ® | Amgen | CHO cells |
| Panitumumab | | |
| Xolair ® | Genentech Inc., Novartis | CHO cells |
| Omalizumab | Pharmaceuticals Corp. | |
| | Tanox Inc. | |
| Zevalin ® | Biogen Idec., Schering AG | CHO cells |
| Ibritumomab tiuxetan | | |
| Bexxar ® | GlaxoSmithKline | Hybridoma, |
| Tositumomab-I131 | | mammalian |
| Soliris ® | Alexion Pharmaceuticals, Inc | Murine |
| Eculizumab | | myeloma |
| | | cell line |
| Ilaris ® | Novartis Pharmaceuticals Corp. | Murine |
| Canakinumab | | Sp2/0-Ag14 |
| | | fused |
| | | hybridoma |
| | | cell line |
| Mylotarg ® | Wyeth Pharmaceuticals | NS0 mouse |
| Gemtuzumab | | myeloma cells |
| ozogamicin | | |
| Arzerra ® | GlaxoSmithKline | NSO mouse |
| Ofatumumab | | myeloma cells |
| Synagis ® | Abbott Laboratories, | NSO mouse |
| Palivizumab | MedImmune Inc. | myeloma cells |
| Tysabri ® | Élan Pharmaceuticals, | NSO mouse |
| Natalizumab | Biogen Idec. | myeloma cells |
| Erbitux ® | ImClone Systems | Sp2/0 mouse |
| Cetuximab | Merck & Co., Inc., Bristol- | myeloma cells |
| | Myers Squibb | |
| Remicade ® | Centocor Ortho Biotech Inc. | Sp2/0 mouse |
| Infliximab | | myeloma cells |
| Reopro ® | Centocor Ortho Biotech Inc., | Sp2/0 mouse |
| Abciximab | Eli Lilly & Co. | myeloma cells |
| Simulect ® | Novartis Pharmaceuticals Corp. | Sp2/0 mouse |
| Basiliximab | | myeloma cells |
| Zenapax ® | F. Hoffmann-La Roche Ltd., | Sp2/0 mouse |
| Daclizumab | PDL (Protein Design Labs) | myeloma cells |
| | BioPharma | |

Fc-Fusion Proteins

| | | |
|---|---|---|
| Amevive ® | Astellas Pharma Inc. | CHO cells |
| Alefacept | | |
| Arcalyst ® | Regeneron Pharmaceuticals Inc. | CHO cells |
| Rilonacept | | |
| Enbrel ® | Amgen, Wyeth Pharmaceutical | CHO cells |
| Etanercept | | |
| Orencia ® | Bristol-Myers-Squibb | CHO cells |
| Abatacept | | |

Hormones

| | | |
|---|---|---|
| Follistim ® | Schering-Plough Corp. | CHO cells |
| Follitropin beta | | |
| Gonal-F ® | EMD Serono, Inc. | CHO cells |
| Follitropin alfa | | |
| Luveris ® | EMD Serono, Inc. | CHO cells |
| Luteinizing hormone | | |
| OP-1 Putty | Stryker Biotech | CHO cells |
| Osteogenic Protein-1 | | |
| (BMP-7) | | |
| Ovidrel ® | EMD Serono, Inc. | CHO cells |
| Choriogonadotropin α | | |
| Thyrogen ® | Genzyme Corp | CHO cells |
| Thyrotropin alfa | | |
| Serostim ®, Saizen ®, | EMD Serono, Inc. | Murine cell |
| Zorbtive ™ | | line (mouse |
| Somatropin | | C127) |

Cytokines

| | | |
|---|---|---|
| Aranesp ® | Amgen | CHO cells |
| Darbepoetin alfa | | |
| Avonex ® | Biogen Idec, Inc. | CHO cells |
| Interferon beta-1a | | |
| Neorecormon ® | Hoffmann-La Roche Ltd. | CHO cells |
| Epoetin beta | | |
| Procrit ®, Epogen ® | Amgen, Centocor Ortho | CHO cells |
| Epoetin alfa | Biotech Inc. | |
| Rebif ® | Pfizer, Inc., EMD Serono, Inc. | CHO cells |
| Interferon beta-1a | | |

Clotting Factors

| | | |
|---|---|---|
| Helixate FS | ZLB Behring | BHK cells |
| Coagulation | | |
| factor VIII | | |
| Kogenate FS | Genentech | BHK cells |
| Coagulation | | |
| factor VIII | | |
| NovoSeven ®, | Novo Nordisk | BHK cells |
| Coagulation | | |
| Factor VIIa | | |
| Advate ® | Baxter International Inc. | CHO cells |
| Antihemophilic factor | | |
| BeneFIX ® | Wyeth Pharmaceuticals | CHO cells |
| Coagulation | | |
| Factor IX | | |
| ReFacto ® | Wyeth Pharmaceuticals | CHO cells |
| Antihemophilic Factor | | |
| Xyntha ® | Wyeth Pharmaceuticals | CHO cells |
| Coagulation | | |
| factor VIII | | |
| Xigris ® | Eli Lilly & Co. | HEK293 |
| Drotrecogin alfa | | |
| (Activated Protein C) | | |

Enzymes

| | | |
|---|---|---|
| Activase ®, Cathflo | Genentech, Boehringer | CHO cells |
| Activase ®, | Ingelheim Pharma KG | |
| Actilyse ® | | |
| Alteplase | | |
| Aldurazyme ® | Genzyme Corp | CHO cells |
| Laronidase | | |
| Cerezyme ® | Genzyme Corp. | CHO cells |
| Imiglucerase | | |
| Fabrazyme ® | Genzyme Corp | CHO cells |
| agalsidase-β | | |
| Hylenex ®, | MediCult A/S, MidAtlantic | CHO cells |
| Cumulase ® | Diagnostics, Inc., Halozyme | |
| Hyaluronidase | Baxter Healthcare | |
| Myozyme ® | Genzyme Corp | CHO cells |
| Alglucosidase alfa | | |
| Naglazyme ® | BioMarin Pharmaceutical Inc. | CHO cells |
| N-acetylgalactosamine | | |
| 4-sulfatase | | |
| Pulmozyme ® | Genentech, Hoffmann-La | CHO cells |
| Human DNase | Roche Ltd. | |
| TNKase ® | Genentech | CHO cells |
| Tenecteplase | | |
| Elaprase ® | Shire Pharmaceuticals | human cell |
| Idursulfase | | line (HT-1080) |

TABLE 2
FDA-APPROVED GLYCOSYLATED POLYPEPTIDES AND
CELL SOURCES FOR PRODUCTION
SORTED BY CELL LINE DERIVATION

| | | |
|---|---|---|
| Agent | Vendor | Cell Source |

Human Hek293 cell-derived

| | | |
|---|---|---|
| Xigris ® | Eli Lilly & Co. | HEK293 |
| Drotrecogin alfa | | |
| (Activated Protein C) | | |

Human HT-1080 cell-derived

| | | |
|---|---|---|
| Elaprase ® | Shire Pharmaceuticals | human cell |
| Idursulfase | | line (HT-1080) |
| CHO cell derived | | |
| Actemra ® | Genentech Inc. | CHO cells |
| Tocilizumab | | |
| Avastin ® | Genentech Inc., | CHO cells |
| Bevacizumab | Hoffmann-La Roche | |
| | Ltd. | |
| Campath ® (US), | Genzyme Corp. | CHO cells |
| Mabcampath ® (EU) | | |
| Alemtuzumab | | |
| Herceptin ® | F. Hoffmann-La Roche Ltd, | CHO cells |
| Trastuzumab | Genentech Inc. | |
| Humira ® | Abbott Laboratories | CHO cells |
| Adalimumab | | |
| Rituxan ® | Genentech | CHO cells |
| Rituximab | | |
| Simponi ® | Centocor Ortho Biotech Inc., | CHO cells |
| Golimumab | Johnson & Johnson Co., | |
| | Schering-Plough Corp. | |
| Stelara ™ | Centocor Ortho Biotech Inc. | CHO cells |
| Ustekinumab | | |
| Vectibix ® | Amgen | CHO cells |
| Panitumumab | | |
| Xolair ® | Genentech Inc., Novartis | CHO cells |
| Omalizumab | Pharmaceuticals Corp. | |
| | Tanox Inc. | |
| Zevalin ® | Biogen Idec., Schering AG | CHO cells |
| Ibritumomab tiuxetan | | |
| Amevive ® | Astellas Pharma Inc. | CHO cells |
| Alefacept | | |
| Arcalyst ® | Regeneron Pharmaceuticals Inc. | CHO cells |
| Rilonacept | | |
| Enbrel ® | Amgen, Wyeth Pharmaceutical | CHO cells |
| Etanercept | | |
| Orencia ® | Bristol-Myers-Squibb | CHO cells |
| Abatacept | | |
| Follistim ® | Schering-Plough Corp. | CHO cells |
| Follitropin beta | | |
| Gonal-F ® | EMD Serono, Inc. | CHO cells |
| Follitropin alfa | | |
| Luveris ® | EMD Serono, Inc. | CHO cells |
| Luteinizing hormone | | |
| OP-1 Putty | Stryker Biotech | CHO cells |
| Osteogenic Protein-1 | | |
| (BMP-7) | | |
| Ovidrel ® | EMD Serono, Inc. | CHO cells |
| Choriogonadotropin α | | |
| Thyrogen ® | Genzyme Corp | CHO cells |
| Thyrotropin alfa | | |
| Aranesp ® | Amgen | CHO cells |
| Darbepoetin alfa | | |
| Avonex ® | Biogen Idec, Inc. | CHO cells |
| Interferon beta-1a | | |
| Neorecormon ® | Hoffmann-La Roche Ltd. | CHO cells |
| Epoetin beta | | |
| Procrit ®, Epogen ® | Amgen, Centocor Ortho | CHO cells |
| Epoetin alfa | Biotech Inc. | |
| Rebif ® | Pfizer, Inc., EMD Serono, Inc. | CHO cells |
| Interferon beta-1a | | |
| Advate ® | Baxter International Inc. | CHO cells |
| Antihemophilic factor | | |
| BeneFIX ® | Wyeth Pharmaceuticals | CHO cells |
| Coagulation Factor IX | | |
| ReFacto ® | Wyeth Pharmaceuticals | CHO cells |
| Antihemophilic Factor | | |
| Xyntha ® | Wyeth Pharmaceuticals | CHO cells |
| Coagulation | | |
| factor VIII | | |
| Activase ®, Cathflo | Genentech, Boehringer | CHO cells |
| Activase ®, | Ingelheim Pharma KG | |
| Actilyse ® | | |
| Alteplase | | |
| Aldurazyme ® | Genzyme Corp | CHO cells |
| Laronidase | | |
| Cerezyme ® | Genzyme Corp. | CHO cells |
| Imiglucerase | | |
| Fabrazyme ® | Genzyme Corp | CHO cells |
| agalsidase-β | | |
| Hylenex ®, | MediCult A/S, MidAtlantic | CHO cells |
| Cumulase ® | Diagnostics, Inc., Halozyme | |
| Hyaluronidase | Baxter Healthcare | |
| Myozyme ® | Genzyme Corp | CHO cells |
| Alglucosidase alfa | | |
| Naglazyme ® | BioMarin Pharmaceutical Inc. | CHO cells |
| N-acetylgalactosamine | | |
| 4-sulfatase | | |
| Pulmozyme ® | Genentech, Hoffmann-La Roche | CHO cells |
| Human DNase | Ltd. | |
| TNKase ® | Genentech | CHO cells |
| Tenecteplase | | |

BHK cell-derived

| | | |
|---|---|---|
| Helixate FS | ZLB Behring | BHK cells |
| Coagulation | | |
| factor VIII | | |
| Kogenate FS | Genentech | BHK cells |
| Coagulation | | |
| factor VIII | | |
| NovoSeven ®, | Novo Nordisk | BHK cells |
| Coagulation | | |
| Factor VIIa | | |

Myeloma/Hybridoma cell-derived

| | | |
|---|---|---|
| Bexxar ® | GlaxoSmithKline | Hybridoma, |
| Tositumomab-I131 | | mammalian |
| Soliris ® | Alexion Pharmaceuticals, Inc | Murine |
| Eculizumab | | myeloma |
| | | cell line |
| Ilaris ® | Novartis Pharmaceuticals Corp. | Murine |
| Canakinumab | | Sp2/0-Ag14 |
| | | fused |
| | | hybridoma |
| | | cell line |
| Mylotarg ® | Wyeth Pharmaceuticals | NS0 mouse |
| Gemtuzumab | | myeloma cells |
| ozogamicin | | |
| Arzerra ® | GlaxoSmithKline | NSO mouse |
| Ofatumumab | | myeloma cells |
| Synagis ® | Abbott Laboratories, | NSO mouse |
| Palivizumab | MedImmune Inc. | myeloma cells |
| Tysabri ® | Élan Pharmaceuticals, | NSO mouse |
| Natalizumab | Biogen Idec. | myeloma cells |
| Erbitux ® | ImClone Systems | Sp2/0 mouse |
| Cetuximab | Merck & Co., Inc., | myeloma cells |
| | Bristol-Myers Squibb | |
| Remicade ® | Centocor Ortho Biotech Inc. | Sp2/0 mouse |
| Infliximab | | myeloma cells |
| Reopro ® | Centocor Ortho Biotech Inc., | Sp2/0 mouse |
| Abciximab | Eli Lilly & Co. | myeloma cells |
| Simulect ® | Novartis Pharmaceuticals Corp. | Sp2/0 mouse |
| Basiliximab | | myeloma cells |
| Zenapax ® | F. Hoffmann-La Roche Ltd., | Sp2/0 mouse |
| Daclizumab | PDL (Protein Design Labs) | myeloma cells |
| | BioPharma | |

Other murine cell line-derived

| | | |
|---|---|---|
| Serostim ®, Saizen ®, | EMD Serono, Inc. | Murine cell |
| Zorbtive ™ | | line (mouse |
| Somatropin | | C127) |

In addition, glycosylated polypeptides currently produced in non-animal cells (e.g. E. coli and yeast), can be produced by methods described herein that affords the glycosylated polypeptides human-like glycosylation patterms that are substantially reduced in Neu5Gc. Examples of recombinant human polypeptides produced in non-animal cells, and therefore lacking human-like glycosylation patterns, which could be produced in animal or human cells to obtain human-like glycosylation patterns are provided in Table 3.

TABLE 3
FDA-APPROVED GLYCOSYLATED POLYPEPTIDES PRODUCED
IN NON-ANIMAL CELLS AND CELL SOURCES FOR PRODUCTION
SORTED BY FUNCTION

| | | |
|---|---|---|
| Agent | Vendor | Cell Source |

Monoclonal Antibodies

| | | |
|---|---|---|
| Cimzia ® | UCB | E. coli |
| Certolizumab pegol | | |
| Lucentis ® | Genentech Inc., Novartis | E. coli |
| Ranibizumab) | Pharmaceuticals Corp. | |

Fc-Fusion Proteins

| | | |
|---|---|---|
| Nplate ® | Amgen | E. coli |
| Romiplostim | | |

Hormones

| | | |
|---|---|---|
| Genotropin ® | Pfizer, Inc. | E. coli |
| Somatropin | | |
| Humanotrope ® | Eli Lilly $ Co | E. coli |
| Somatropin | | |
| Humatrope ® | Eli Lilly & Co. | E. coli |
| Somatropin | | |
| Kepivance ® | Biovitrum AB | E. coli |
| (palifermin) | | |
| keratinocyte growth | | |
| factor | | |
| Norditropin ® | Novo Nordisk | E. coli |
| Somatropin | Pharmaceuticals, Inc. | |
| Nutropin ® | Genentech Inc | E. coli |
| Somatropin | | |
| Omnitrope ® | Novartis Pharmaceuticals Corp. | E. coli |
| Somatropin | | |
| Regranex ® | Johnson & Johnson Co. | Saccharomyces |
| Platelet-derived | | cerevisiae |
| growth factor | | (yeast) |
| (PDGF)-BB | | |
| Fortical ® | Upsher Smith Laboratories | E. coli |
| Calcitonin (salmon) | | |

Cytokines

| | | |
|---|---|---|
| Actimmune ® | Intermune Inc. | E. coli |
| Interferon gamma-1b | | |
| Betaseron ® | Bayer HealthCare | E. coli |
| Interferon beta-1b | Pharmaceuticals | |
| Extavia ® | Novartis Pharmaceuticals Corp. | E. coli |
| Interferon beta-1b | | |
| Infergen ® | Three Rivers Pharmaceuticals | E. coli |
| Interferon alfacon-1 | | |
| Intron ® A | Merck & Co | E. coli |
| Interferon alfa-2b | | |
| Kineret ® (anakinra) | Biovitrum AB | E. coli |
| interleukin-1 receptor | | |
| antagonist (IL-1Ra) | | |
| Neulasta ® | Amgen | E. coli |
| Pegfilgrastim | | |
| Neumega ® | Wyeth Pharmaceuticals | E. coli |
| Des-Pro Interleukin-11 | | |
| Neupogen ® | Amgen, Hoffmann-La | E. coli |
| Recombinant G-CSF | Roche Ltd. | |
| Ontak ® | Eisai Co., Ltd. | E. coli |
| (denileukin diftitox) | | |
| IL-2/diphtheria toxin | | |
| fusion protein | | |
| Pegasys ® | Hoffmann-La Roche Ltd. | E. coli |
| Peginterferon alfa-2a | | |
| Pegintron ® | Merck & Co | E. coli |
| Peginterferon alfa-2b | | |
| Proleukin ® | Novartis Pharmaceuticals Corp. | E. coli |
| Aldesleukin (IL-2) | | |
| Roferon A ® | Hoffmann-La Roche Ltd. | E. coli |
| Interferon alfa-2a | | |

Enzymes

| | | |
|---|---|---|
| Xiaflex ® | Auxilium Pharmaceuticals Inc. | Clostridium |
| Collagenase | | Histolyticum |
| Elitek ® | Sanofi Aventis | Saccharomyces |
| Rasburicase | Pharmaceuticals, Inc. | cerevisiae |
| | | (yeast) |

In practicing the present invention, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonuchotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively. Methods to detect and measure levels of polypeptide gene expression products (i.e., gene translation level) are well-known in the art and include the use polypeptide detection methods such as antibody detection and quantification techniques. (See also, Strachan & Read, Human Molecular Genetics, Second Edition. (John Wiley and Sons, Inc., NY, 1999)).

The compositions of the invention can be used for the same medical indications as the corresponding Neu5Gc-contaminated glycosylated polypeptide or improperly glycosylated polypeptide (having a non-human glycosylation pattern or composition), but with a lowered immune intolerance in the human subject and/or with a potentially higher efficacy due to a lowered circulatory clearance rate. Methods for detecting immune tolerance are well known in the art. For example, the interaction of glycosylated polypeptides wherein Neu5Gc has been substantially reduced can be compared to glycosylated polypeptides from non-treated sources by measuring the immunoreactivity of human serum known to have anti-Neu5Gc antibodies.

The relative immunoreactivity as determined above can be used to predict the relative clearance of the glycosylated polypeptide, i.e. a higher relative immunoreactivity predicts that the glycosylated polypeptide would be more rapidly cleared from the bloodstream and, therefore, more likely to have a lowered bioactivity. Consequently, glycosylated polypeptides wherein Neu5Gc has been substantially reduced will therefore likely have a longer clearance rate than the correlative Neu5Gc-contaminated glycosylated polypeptide and therefore would be predicted to have higher bioactivity. The relative effect of Neu5Gc reduction on plasma clearance in humans can be assessed by, for example, (1) comparing the plasma clearance of glycosylated polypeptides wherein Neu5Gc has been substantially reduced versus glycosylated polypeptides from untreated sources or (2) comparing anti-Neu5Gc antibody levels wherein Neu5Gc has been substantially reduced versus glycosylated polypeptides from untreated sources, such as in a CMAH negative animal model, such as the CMAH null mouse.

Compounds described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternately, compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion, or an aqueous or non-aqueous suspension. For rectal administration, the invention compounds may be combined with excipients such as cocoa butter, glycerin, gelatin or polyethylene glycols and molded into a suppository.

Compounds may be formulated to include other medically useful drugs or biological agents. The compounds also may be administered in conjunction with the administration of other drugs or biological agents useful for the disease or condition to which the invention compounds are directed.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused.

A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### EXAMPLES

### Example 1

### Materials and Methods

Mice. Cmah null mice⁶, and were backcrossed to C57Bl/6 mice for >10 generations.

Sialidase Treatment of Cet and Pan. One mg each of Cetuximab (Cet) (Erbitux® Bristol-Myers Squibb Co., New York, N.Y.) or Panitumumab (Pan) Vectibix® (Panitumumab, Amgen, Thousand Oaks, Calif.) were treated with 50 mU of active or heat-inactivated Arthrobacter ureafaciens sialidase (EY Laboratories, San Mateo, Calif.) in 100 mM sodium acetate, pH 5.5, at 37° C. for 24 h. Samples were used for ELISA or Western Blots.

Periodate Treatment of Cet and Pan. ELISA plates were coated with untreated Cetuximab and Panitumumab (1 μg/well), then blocked with PBST (137 mM NaCl, 2.7 mM KCl, 10 mM Na2HPO4, 1.76 mM KH2PO4, pH 7.4 containing 0.1% Tween-20) for 2 h and incubated with freshly made 2 mM sodium metaperiodate in PBS for 20 min at 4° C. in the dark. The reaction was stopped using 200 mM sodium borohydride to a final concentration of 20 mM. As a control, periodate and borohydride were pre-mixed and then added to the wells (the borohydride inactivates the periodate). To remove resulting borates, wells were then washed 3 times with 100 mM sodium acetate, 100 mM NaCl, pH 5.5 before further analysis.

ELISA Detection of Neu5Gc on Cetuximab and Panitumumab. Wells were coated with 1 μg of Cetuximab or Panitumumab (either pre-sialidase treated or post-periodate treated), blocked with TBST for 2 h, and then incubated with affinity-purified chicken anti-Neu5Gc IgY as described⁵ or control IgY (Jackson ImmunoResearch Laboratories, Inc., West Grove, Pa.) for 1 h (1:20,000 in TBST). Binding of IgY was detected using HRP-conjugated donkey anti-chicken IgY antibody (Jackson ImmunoResearch Laboratories, Inc., West Grove, Pa.) (1:50,000 in TBST) and development with O-phenylenediamine in citrate-phosphate buffer, pH 5.5, with absorbance being measured at 495 nm. ELISA samples were studied at least in triplicate. Similar to the ELISA with the anti-Neu5Gc chicken IgY, human anti-Neu5Gc IgG that had been purified from the serum of healthy humans⁷ and biotinylated was also used as the primary antibody (1:100 in TBST). Binding of the human antibodies to the therapeutic antibodies was detected using HRP-conjugated Streptavidin (1:10,000) (Jackson ImmunoResearch Laboratories, Inc., West Grove, Pa.) followed by development as described above. Samples were studied in triplicate.

Western Blot detection of Neu5Gc on Cetuximab and Panitumumab. Cetuximab and Panitumumab (1 μg per lane) were separated by 12.5% SDS-PAGE, and Coomassie stained or blotted on nitrocellulose membranes. Blotted membranes were blocked with TBST containing 0.5% cold water fish skin gelatin overnight at 4° C. and subsequently incubated with affinity-purified chicken anti-Neu5Gc IgY for 4 h at room temperature (1:100,000 in TBST). Binding of the chicken anti-Neu5Gc IgY was detected using an HRP-conjugated donkey anti-chicken IgY antibody for 1 h (1:50,000 in TBST), followed by incubation with SuperSignal West Pico Substrate (Thermo Fisher Scientific, Waltham, Mass.) as per manufacturer's recommendation, exposed to X-ray film and the film developed. Similar to the Western blot with the chicken anti-Neu5Gc IgY, purified biotinylated human anti-Neu5Gc IgG was also used as the primary antibody (1:100 in TBST). Binding of the human antibodies to the therapeutic antibodies was detected using HRP-conjugated Streptavidin (1:10,000 in TBST) followed by development as described above.

CIC-C1q Binding Assay. Immune complex formation was detected by using the CIC (C1Q) ELISA Kit (BÜHLMANN Laboratories AG, Schonenbuch, Switzerland) as described in the manufacturers's guidelines⁸. Briefly, 100 μl of human serum with low or high anti-Neu5Gc antibodies (S30 and S34, respectively, from Ref. 7) was incubated with 40 μg of Cetuximab or Panitumumab for 14 h at 4° C. 1:50 dilutions of the mix were applied to human C1 q coated ELISA wells, and incubated for 1 h at 25° C. Binding was detected using alkaline phosphatase conjugated Protein A. After another washing step, the enzyme substrate (para-nitrophenyl-phosphate) was added followed by a stopping step. The absorbance was measured at 405 nm. Samples were studied in triplicate.

DMB-HPLC. Samples were subjected to base treatment with 0.1 M NaOH (final) at 37° C. for 30 min to remove O-acetyl esters, and the sialic acids were released by acid hydrolysis with 2 M acetic acid (final), at 80° C. for 3 hours. Samples were passed through Microcon-10 columns (20 min, 10000×g; Millipore), derivatized with 1,2-diamino-4,5-methyl dioxybenzene (DMB), and analyzed by HPLC as described⁹ in Hara et al., 1986, J. Chromatography 377:111-119. Measured values of non acid-treated controls (free sialic acids) were subtracted.

Generation of murine anti-Neu5Gc antibodies. Haemophilus influenzae strain 201935 were grown to mid-log in sialic acid-free media (RPMI 1640 media (Sigma-Aldrich, St. Louis, Mo.) supplemented with 1 μg/ml protoporphyrin IX (Sigma-Aldrich, St. Louis, Mo.), 1 μg/ml L-histidine (Sigma-Aldrich, St. Louis, Mo.), 10 μg/ml beta-Nicotinamide adenine dinucleotide (Sigma-Aldrich, St. Louis, Mo.), 0.1 mg/ml hypoxanthine (Sigma-Aldrich, St. Louis, Mo.), 0.1 mg/ml uracil (Sigma-Aldrich, St. Louis, Mo.), and 0.8 mM sodium pyruvate (Invitrogen, Carlsbad, Calif./USA) 36 with or without addition of 1 mM Neu5Gc, heat-killed, and injected intraperitoneally (200 μl of OD600 nm=0.4) into Cmah null mice.

Kinetics of Cetuximab and Panitumumab in mice with a human-like Neu5Gc-deficiency, in the absence or presence of anti-Neu5Gc antibodies. Cetuximab or Panitumumab in PBS (0.24 μg per gram mouse body weight) were injected i.v. and 14 h later, mouse serum pooled from syngeneic Cmah null mice containing anti-Neu5Gc antibodies (or pooled serum from syngeneic naïve or control immunized mice) was passively transferred via IP injection into syngeneic Cmah null mice. Mice were bled 0, 2, 8, 32, 56 and 80 h after the passive transfer of mouse serum. For quantification of therapeutic antibodies concentration in the sera, wells of ELISA plates were coated with 1 μg of anti-human IgG (Biorad, Hercules, Calif.), then blocked with TBST for 2 h and incubated with 1:500 dilutions of the sera per well. Captured therapeutic antibodies were detected by HRP-conjugated anti-human Fc antibody (1:10,000) (Jackson, ImmunoResearch Laboratories, Inc., West Grove, Pa.), with development by O-phenylenediamine in citrate-phosphate buffer, pH 5.5, and absorbance measured at 495 nm (n=5 for injections of both control sera groups, n=10 for injections of anti-Neu5Gc serum groups).

### Quantification of Anti-Neu5Gc IgG Antibodies in Neu5Gc-Immunized Mice.

A Neu5Gcα2-6Galβ1-4Glc-conjugate⁷ (1 μg/well) and serial dilutions of mouse IgG as standards (0.625-20 ng/well) were used for coating overnight, then blocked with PBST for 2 h and incubated with pooled serum from Neu5Gc immunized mice (1:250 dilution) for 2 h at 25° C. Binding of mouse IgG was detected by using HRP-conjugated goat anti-mouse IgG-Fc (Jackson ImmunoResearch Laboratories, Inc.; West Grove, Pa.) (1:10,000 in PBST) and development with O-phenylenediamine in citrate-phosphate buffer, pH 5.5, with absorbance being measured at 490 nm. ELISA samples were studied in triplicate.

Levels of anti-Neu5Gc IgG in mice with a human-like Neu5Gc-deficiency after injections of the therapeutic antibodies, or murine IgG. Cmah null mice were injected i.v. with 4 μg antibody/gram mouse body weight in PBS weekly for three weeks. Mice were bled initially, and again one week after the third i.v. injection. Wells of ELISA plates were coated with 1/1000 dilutions of human (Neu5Gc-deficient) or chimpanzee (Neu5Gc-positive) serum glycoproteins (Note that the only major difference between human and chimp serum glycosylation is the absence or presence of Neu5Gc¹⁰). Alternatively, wells were coated with human or bovine fibrinogen, which carry Neu5Ac or Neu5Gc on otherwise identical N-glycans¹¹. Wells were then blocked with TBST for 2 h followed by incubation with 1:100 dilutions of the mouse sera. Binding of the mouse antibodies was detected by using HRP-conjugated goat anti-mouse IgG Fc fragment antibody (1:10000 in TBST). Neu5Gc-specific binding (change in OD495 nm) was determined by subtracting the background signal of the wells coated with Human serum or Human fibrinogen (no Neu5Gc) from the signal with Chimpanzee serum or Bovine fibrinogen (Neu5Gc-containing) coated wells. Data were obtained in triplicate (n=5 for injection of mIgG, n=4 for injection of Panitumumab, n=6 for injection of Cetuximab).

Reduction of Neu5Gc Contamination in Glycosylated Polypeptides in 293T Human Kidney Cells. 293T human kidney cells were grown in DME supplemented with 10% FCS. Cells were lifted from culture plate using 20 mM EDTA in PBS and allowed to grow to 50% confluence. Buffered 100 mM Neu5Gc was added to the culture in duplicate at a final 5 mM concentration, and the cells were grown in this supplemented media for 3 days. At the end of this Neu5Gc pulse, the cells were once again lifted using 20 mM EDTA in PBS, pelleted, washed once with PBS to remove any excess Neu5Gc and then suspended in 30 ml of growth medium. Five ml of this cell suspension was added to each of 5 P-100 dishes. The last aliquot of cell suspension, time “0”, was immediately harvested by pelleting the cells, washing once with PBS, followed by suspending the cells in 1 ml of PBS and transferring to a 1.5 ml microcentrifuge tube. The cells were re-pelleted and frozen until all time points were collected. Buffered 100 mM Neu5Ac (Inalco spa, Milan, Italy) was added to each of the other 5 plates for the “Neu5Ac chase”, and an equivalent amount of media added to the “minus chase” samples. The cells were harvested at day 1, 2, 3, 4 and 5 by scraping into the culture media, collecting by pelleting, washing once with PBS, transferring to a 1.5 ml microcentrifuge tube, pelleting and freezing the cell pellet. At the end of the 5 days of chase, all collected cell pellets were homogenized in 300 μl of ice-cold 20 mM potassium phosphate pH 7 using 3-20 sec burst with a Fisher Sonicator. Glycoconjugate-bound Sias (sialic acids) were precipitated by adding 700 μl of 100% ice-cold ethanol (final 70% ethanol) and incubating at −20° C. overnight. The samples were spun at 20000×g for 15 min and the supernatants transferred to clean tubes and dried on a speed vac. The precipitated glycoconjugates and dried ethanol supernatants were each suspended in 100 μl of 20 mM potassium phosphate pH 7 by sonication. Sias were released from both fractions by acid hydrolysis with 2 M acetic acid (final) and incubating at 80° C. for 3 h. Samples were passed through a Microcon-10 filter and the filtrate derivatized with DMB reagent, for analysis of Sias by HPLC.

Reduction of Neu5Gc Contamination in Glycosylated polypeptides in CHO Cells. A similar approach was taken to CHO cells stably expressing a Siglec-Fc protein in the medium, except that the Neu5Gc pulse was omitted, and the secreted glycoproteins were captured on Protein-A Sepharose beads. The cells were also processed similarly, except that total cell membranes were pelleted by centrifugation. The Sias content of the secreted proteins and cell membranes was determined by acid hydrolysis, DMB derivatization and HPLC. The cell membranes were also studied for NueSGc content by Western-blotting with the chicken anti-Neu5Gc IgY, as described above.

### Comparison of Cetuximab and Panitumumab

Two FDA-approved antibodies with the same therapeutic target, the EGF receptor, were compared: Erbitux® (Cetuximab), a chimeric antibody produced in mouse myeloma cells^{12, 13}, and Vectibix® (Panitumumab), a fully human antibody produced in Chinese Hamster Ovary cells (CHO cells)¹⁴. The samples studied were preparations that would normally be administered to patients.

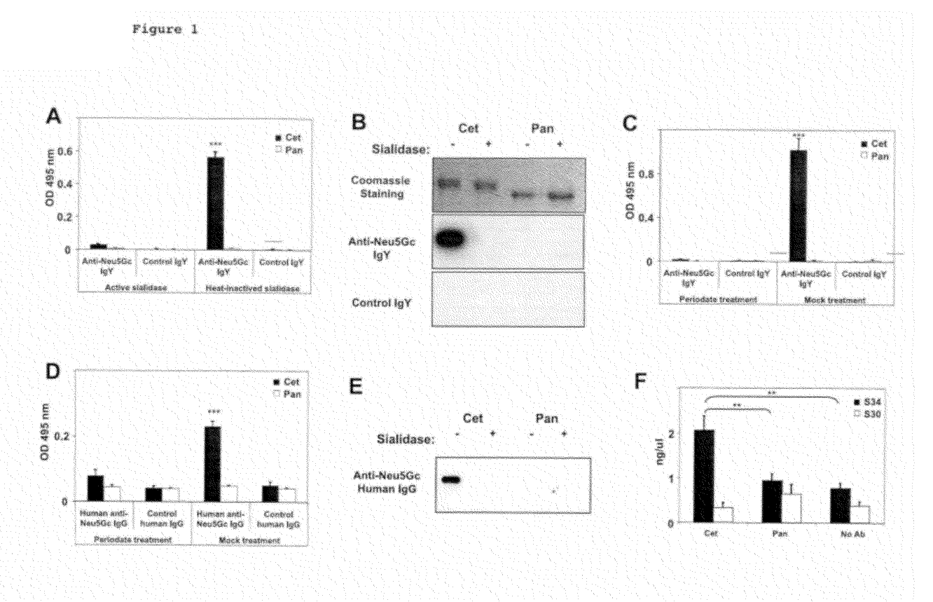

ELISA We first did ELISA assays using an affinity-purified polyclonal chicken anti-Neu5Gc antibody preparation that is highly mono-specific for Neu5Gc⁵, alongside a non-reactive control IgY. Bound Neu5Gc was easily detectable on Cetuximab, and not on Panitumumab (FIG. 1A), and sialidase pretreatment abolished binding, confirming specificity.

Western Blot. Western Blot analysis also showed anti-Neu5Gc IgY reactivity on the Cetuximab heavy chain, but not on Panitumumab (FIG. 1B).

Sialic Acid Specificity of Anti-Neu5Gc IgY Binding. Sialic acid-specificity of anti-Neu5Gc IgY binding was reaffirmed by pre-treatment with mild sodium periodate, under conditions that selectively cleave Sia side chains (FIG. 1C), and abolish reactivity of such antibodies^{15,5}. Cet and Pan were used for coating, then blocked, and sialic acid epitopes eliminated chemically using sodium metaperiodate. The reaction was stopped with sodium borohydride. As a control, periodate and borohydride were pre-mixed and then added to the wells (the borohydride inactivates the periodate). ELISA samples were studied at least in triplicate and data shown are Mean+/−SD. ***p<0.001, Paired Two-tailed t-test.

Sialic Acid Quantity. The levels of Sialic acids on the therapeutic antibodies (TAbs) Cet and Pan were determined. Cet and Pan were treated with sialidase or heat-inactivated sialidase as for FIG. 1A and used for coating ELISA wells, then blocked and incubated with human anti-Neu5Gc IgG that had been purified from the serum of healthy humans and biotinylated (FIG. 1D). Samples were studied in triplicate and data shown as Mean+/−SD. ***p<0.001 Panitumumab carries 0.22 mol/mol of Sias, with <0.1% Neu5Gc. In contrast, Cetuximab carries 1.84 mol/mol of Sias, mostly as Neu5Gc. Cet and Pan (1 μg each) were separated by SDS-PAGE and Coomassie stained or blotted (see FIG. 1B). Neu5Gc content was detected by using biotinylated human anti-Neu5Gc IgG (FIG. 1E). Immune complex formation with Cet or Pan in whole human serum was detected using the CIC (C1Q) ELISA Kit (BÜHLMANN Laboratories AG, Schönenbuch/Switzerland) as described in the manufacturer's guidelines (FIG. 1F). The absorbance was measured at 405 nm. Samples were studied in triplicate and data are shown as Mean+/−SD. **p<0.01, Paired Two-tailed t-test. Cetuximab formed immune complexes in a human serum with high levels of anti-Neu5Gc antibodies (serum S34, from reference 7), and not with the low titer serum (serum S30, from reference 7). In contrast, Panitumumab gave no detectable immune complex formation with either sera.

Effect of Neu5Gc on Glycosylated Polypeptide Circulatory Clearance Rate. The effect of Neu5Gc on clearance rate when circulating anti-Neu5Gc antibodies are present was evaluated. To simulate the effect of Neu5Gc sialic acids on therapeutic protein elimination in humans, a mouse model with a human-like defect in the Cmah gene, which encodes the enzyme that generates activated Neu5Gc (CMP-Neu5Gc)³, was used. Such mice can make anti-Neu5Gc antibodies upon immunization with glycosidically-bound but not free Neu5Gc^{3,16}. To most closely mimic clearance behavior in a human, the mice were immunized with a Neu5Gc-loaded microbe as described¹⁷.

Cet or Pan were injected i.v., to obtain a concentration of 1 μg/ml in extracellular fluid volume (ECF) according to mouse body weight¹⁸. Sera pooled from naïve, control-immunized or Neu5Gc-immunized syngeneic mice were passively transferred via intraperitoneal injection, ensuring equal starting concentrations of circulating anti-Neu5Gc antibodies. Anti-Neu5Gc IgG levels in the pooled sera from Neu5Gc-immunized mice were quantified by ELISA with a Neu5Gcα2-6Galβ1-4Glc-conjugate as a target, as previously described⁷ (97.5 μg/ml). The amount of pooled antibody injected was calculated to achieve an approximate starting concentration of 4 μg/ml IgG in the ECF of these mice, i.e. ˜4 times excess of anti-Neu5Gc antibodies compared to the drug in mice, and similar to levels found in some humans⁷. Mice were bled periodically after the passive transfer of mouse serum.

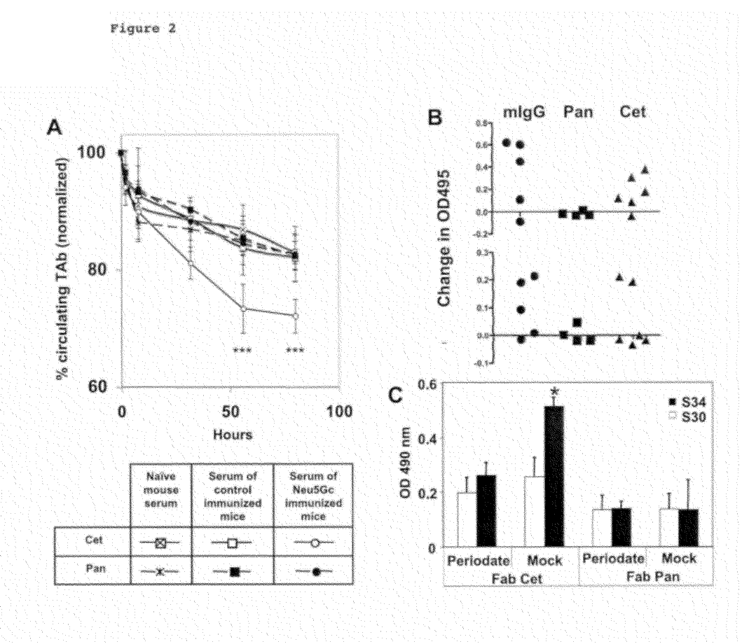

Clearance was monitored by a sandwich ELISA specific for human IgG-Fc (FIG. 2A). Absorbance was measured at 495 nm. The Y axis starts at 60%, in order to better display the difference in kinetics. ***p<0.001, Unpaired Two-tailed t-test. While both drugs had a similar clearance rate in mice pre-injected with serum from naïve or control-immunized mice, Cetuximab showed a significant decrease in circulating levels when anti-Neu5Gc antibodies were pre-injected.

To further simulate the clinical situation, equal amounts of Cetuximab or Panitumumab were i.v. injected weekly into Neu5Gc-deficient Cmah −/− mice in typical human dosages (4 μg/g body weight). The mice were bled at the initial injection and after the 3rd i.v. injection. In order to detect Neu5Gc specific antibodies by ELISA, wells were coated with human (Neu5Gc-deficient) and chimpanzee (Neu5Gc-positive) serum glycoproteins (Upper Panel), or alternatively with human or bovine fibrinogen (Lower Panel) (FIG. 2B). Data were obtained in triplicate. To exclude any impact of the partly (Cetuximab) or fully human protein portion (Panitumumab) in mice, murine IgG was also injected as a positive control, as it happens to carry primarily Neu5Gc. Cetuximab and murine IgG (but not Panitumumab) induced a Neu5Gc-specific IgG immune response (FIG. 2B).

Direct Binding of Anti-Neu5Gc Antibodies to Cet and Pan. Direct binding of anti-Neu5Gc antibodies from whole human sera to the glycosylated polypeptides, Cet and Pan, was evaluated. Fab fragments of Cet and Pan were isolated using the Pierce® Fab Preparation Kit according to the manufacturer's manual. Fab fragments (1 μg/well) were used as target molecules in ELISA. Sialic acid specific binding was determined with sodium metaperiodate treatment. Wells were then blocked and incubated with human sera (S30 and S34 with low and high anti-Neu5Gc IgG titers, respectively, from Ref. 7). Binding of human IgG was detected by using anti-human IgG-Fc. The absorbance was measured at 490 nm and ELISA samples were studied in triplicate (FIG. 2C). p<0.05. Paired Two-tailed t-test. Mild periodate sensitive binding of serum IgG from a high anti-Neu5Gc titer serum (S34 from Ref 7, which had >15 μg/ml of IgG antibodies against Neu5Gcα2-6Galβ1-4Glc−) was detected for the Fab fragments of Cetuximab and not to those of Panitumumab. In contrast, incubation with another human serum containing very low Neu5Gc-antibodies (serum S30 from Ref 7, which had <2 μg/ml of IgG antibodies against Neu5Gcα2-6Galβ1-4Glc−) did not show much periodate-sensitive binding. Thus, whole human sera with high (but not low) titers of anti-Neu5Gc antibodies showed sialic acid dependent (mild periodate sensitive) binding of serum IgG to Cetuximab, but not to Panitumumab.

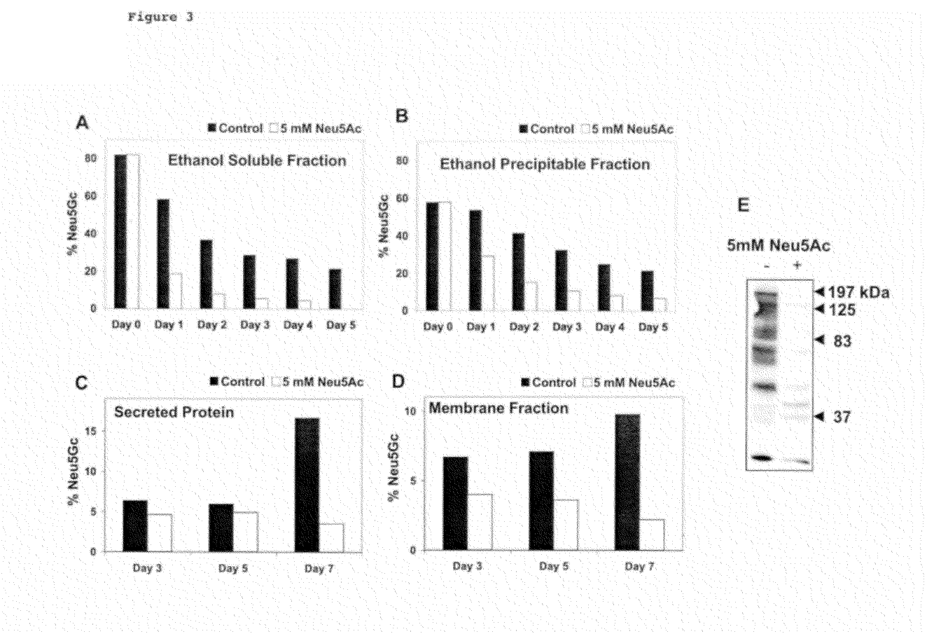

Free Neu5Ac in Human 293T Cell Culture Medium Reduces Neu5Gc Contamination. Human 293T cells were preloaded with Neu5Gc, and then chased in the presence or absence of 5 mM Neu5Ac. Human 293T cells were grown in the presence of 5 mM Neu5Gc for 3 days. The cells were then washed with PBS, split into two identical cultures, and 5 mM Neu5Ac was added to one of the cultures as shown on the graph. Cells were harvested as described above, and the Neu5Gc and Neu5Ac content of both the ethanol soluble (FIG. 3A) and ethanol precipitable proteins (FIG. 3B) was analyzed by HPLC. The percent Neu5Gc shown is the amount of Neu5Gc relative to the total sialic acids. Such Neu5Ac addition resulted in more rapid disappearance of ethanol-precipitable (glycosidically-bound) Neu5Gc from the cells and also from secreted glycoproteins (FIG. 3A-B). Thus, Neu5Ac addition to the medium eliminates or reduces Neu5Gc contamination of human cells.

Free Neu5Ac in Non-Human CHO Cell Culture Medium Reduces Neu5Gc Contamination. However, many recombinant glycosylated polypeptides are currently produced in non-human cells, e.g., Chinese Hamster Ovary (CHO) cell lines. Glycosylated polypeptide produced in CHO cells carry small amounts of Neu5Gc^{19,5}. Feeding Neu5Ac reduces Neu5Gc in CHO cells, both for membrane glycoproteins and for a secreted recombinant protein (FIG. 3 C-E). Stably transfected CHO-KI cells expressing a recombinant soluble IgG-Fc fusion protein were grown in the absence or presence of 5 mM Neu5Ac. The individually collected media was centrifuged to remove cell debris and adjusted to 5 mM Tris-HCl pH 8. The fusion protein was purified using Protein-A Sepharose. Sialic acid content was determined by DMB-HPLC analysis (FIG. 3C). The area under each peak was obtained and the percent of Neu5Gc in each sample was determined relative to Neu5Ac. Total cell membranes from the same CHO cells were prepared and used for DMB-HPLC analysis (FIG. 3D). CHO membrane proteins from the above experiments were separated by SDS-PAGE and transferred onto nitrocellulose membranes (FIG. 3E). The expression of Neu5Gc was detected by incubating with polyclonal affinity purified chicken anti-Neu5Gc antibody.

### Monoclonal Antibodies Specific to Human Epidermal Growth Factor Receptor

Purification of 108 Monoclonal Antibodies. Hybridoma cell lines that express monoclonal antibodies specific to human epidermal growth factor receptor are disclosed in U.S. Pat. No. 6,217,866, incorporated by reference herein, the preparation of which is modified as follows: The 108 IgG2a hybridoma cell line is generated by immunizing mice with CH 71 cells expressing the EGF receptor and cultured in Dulbecco's modified Eagle's medium supplemented with 10% fetal calf serum and 5 mM Neu5Ac depleted of complement activity by incubation at 56° C. for 30 minutes and grown in glutamine, penicillin, streptomycin and sodium pyruvate, at 37° C. in 5% CO2:95% air atmosphere. The 96 IgM hybridoma cell line is generated by the same procedure as that described for the 108 IgG2a hybridoma cell line.

The medium wherein the monoclonal antibodies are secreted is separated from the hybridoma cells. Monoclonal antibodies are precipitated by slow addition of saturated ammonium sulfate at 4° C. to a final concentration of 45% (v/v), pH 7.5, for 24 hours. The precipitate is collected by centrifugation at 10,000 g for 15 minutes and washed twice with 50% v/v ammonium sulfate, pH 7.5. at 4° C. Further purification is carried out by affinity chromatography on Sepharose CL protein A (Pharmacia) in 0.14M Tris buffer, pH 8.0 and the 108 monoclonal antibody is eluted with 0.1M citrate buffer, pH 3.0, followed by extensive dialysis against PBS.

Purification, Specific Activity and Immunoreactivity of F (ab)'2, and F(ab)′ Fragment of 108 Monoclonal Antibody. 108 monoclonal antibody (5 mg/ml) in 0.1M sodium-acetate buffer at pH 3.9 is digested in the presence of 4% w/w pepsin (Worthington Biochemical Corporation, New Jersey) for 7 hours at 37° C. Digestion is terminated by adjusting the pH to 8.0 with 2M Tris, followed by dialysis against PBS at 4° C. Remaining intact IgG molecules are removed by protein A affinity chromatography. The Fc portion and smaller fragments are removed by gel filtration on Sepharose G-100. For the preparation of monovalent Fab′ fragment, the F(ab)′2 (2 mg/ml) is reduced by 10 mM dithiothreitol in 20 mM Tris buffer, pH 8.2, for 1 hour at 37° C. Alkylation is performed in 40 mM iodoacetamide for 30 minutes at 37° C., followed by extensive dialysis against PBS at 4° C. Purity and complete digestion of the various fragments are analyzed by sodium dodecylsulfate polyacrylamide gel electrophoresis (SDS-PAGE). ¹²⁵I-labeling of 108 monoclonal antibody is performed by the chloramine T method (Hunter and Greenwood, Preparation of ¹³¹Iodine Labeled Human Growth Hormone of High Specific Activity, Nature, Vol. 196, 465-6, (1962)). Specific activities of about 3×10⁶ cpm/μg IgG are usually obtained.

Immunoreactivity of the F(ab)′2 and F(ab) fragments of 108 monoclonal antibody are compared to native intact 108 monoclonal antibody in their capacity to compete with the binding of ¹²⁵I labeled 108 to EGF receptors exposed on KB cells.

Human Follicle Stimulating Hormone (hFSH)

The protein dimer of hFSH contains 2 polypeptide units, labeled alpha and beta subunits. The alpha subunits of leutenizing hormone (LH), FSH, thyroid stimulating hormone (TSH), and human chorionic gonadotrophin (hCG) are identical, and contain 92 amino acids. The beta subunits vary. FSH has a beta subunit of 118 amino acids (FSHB), which confers its specific biologic action and is responsible for interaction with the FSH-receptor.

Expression. Construction of expression plasmid pRF375, which expresses the alpha subunit under mouse metallothienine control, and expression plasmid CL28FSH2.8BPV, which expresses the beta subunit, as well as their coexpression in mouse C127 cells is described in U.S. Pat. No. 5,767,251 and is herein incorporated by reference. Glycosylation mutants of hFSH as well as coexpression of alpha and beta subunits in CHO cells are disclosed in U.S. Pat. No. 7,700,112, incorporated by reference herein. hFSH is produced by growing stably transfected cells in two 850 cm² roller bottles. The volume of serum-free production medium (DMEM-F12+IFCS+5 mM Neu5Ac) conditioned in this run is 2600 ml. Quantification of hFSH produced is by DSL Active® FSH ELISA using the conversion 1IU=138 ng FSH.

Purification from Production Media. Production media containing hFSH is harvested and filtered using 0.22 μm filter units and frozen at −70° C. The target proteins in media are thawed overnight at 4° C., and concentrated by ultrafiltration using Ultrasette Screen Channel TFF device, 10 K Omega membrane, P/N 0S010C70 (Pall Life Science). The retentate is recovered and dialyzed overnight versus 0.1 M Tris, pH 7.4 containing 0.5 M NaCl, 3×5 Liters. The dialyzed protein is recovered, 0.22 μm filtered, and purified immediately or stored at −70° C. until purified.

Immunoaffinity Purification (Alternate Purification). Human follicle stimulating hormone is purified by anti FSH immunoaffinity resin B5 (Serobio) containing 2.2 mg anti-FSH antibody per ml of resin. A 10.2 ml bed volume is prepared in 1.5 cm×10 cm OmniFit column. The resin is pre equilibrated with 0.1 M Tris, pH 7.4 containing 0.5 M NaCl. The dialyzed crude protein is loaded at one ml/min. The column is washed sequentially with the five column volumes of 0.1 M Tris, pH 7.4 containing 0.5 M NaCl, five column volumes of 100 mM ammonium bicarbonate, pH 7.6, and the target protein eluted with 18-20 column volumes of 1 M NH4OH. The fractions containing the eluted protein are pooled, neutralized with glacial acetic acid, and concentrated by ultrafiltration with Amicon stirred cell using Amicon YM 10 membrane. The retentate is dialyzed in Pierce Snakeskin dialysis tubing, 10 K MWCO, versus 4×5 liters of water over 24 hours. The dialyzed protein is recovered and concentrated by Centriprep YM 10 to decrease the volume to approximately one ml.

The apparent recovery of purified protein through this single step immunoaffinity process is 31.4-52.9% at 73.8 to 80.6% heterodimer purity with protein concentration determined by amino acid composition analysis and formula weight originally estimated as 35,000 Da from the MALDI-TOF glycoform distribution of subunits. Identity of the protein is confirmed by N-terminal peptide sequencing.

### Human Activated Protein C

Expression. Recombinant human protein C (r-hPC) is produced in Human Kidney 293 cells or in Syrian hamster cell line AV12 by techniques well known to the skilled artisan such as those set forth in Yan, U.S. Pat. No. 4,981,952, Bang, et al., U.S. Pat. No. 4,775,624 and No. 4,992,373, and in Grinnell, et al., 1987, Bio/Technology 5:1189-1192, incorporated by reference herein.

Human Embryonic Kidney Cell Line 293 is available from the American Type Culture Collection under the accession number ATCC CRL 1573 and the adenovirus-transformed Syrian hamster cell line AV12 is available from the American Type Culture Collection under the accession number ATCC CRL 9595. The transformation procedure is described, for example, in U.S. Pat. No. 4,992,373 where the plasmid encoding r-hAPC is transfected into 293 cells, then stable transformants are identified, subcultured and grown in serum-free media supplemented with 5 mM Neu5Ac. After fermentation, cell-free medium was obtained by microfiltration.

Purification. Recombinant human protein C (r-hPC) is purified by methods well known in the art such as by methods described in Carlson, et al., U.S. Pat. No. 6,159,468, herein incorporated by reference.

Human protein C is separated from the culture fluid by an adaptation of the techniques of Yan, U.S. Pat. No. 4,981,952. The clarified medium is made 4 mM in EDTA before it is absorbed to an anion exchange resin (Fast-Flow Q, Pharmacia). After washing with 4 column volumes of 20 mM Tris, 200 mM NaCl, pH 7.4 and 2 column volumes of 20 mM Tris, 150 mM NaCl, pH 7.4, the bound recombinant human protein C zymogen is eluted with 20 mM Tris, 150 mM NaCl, 10 mM CaCl₂, pH 7.4. Purity is judged by SDS-polyacrylamide gel electrophoresis.

Further purification of the protein is accomplished by making the protein 3 M in NaCl followed by adsorption to a hydrophobic interaction resin (Toyopearl Phenyl 650 M, TosoHaas) equilibrated in 20 mM Tris, 3 M NaCl, 10 mM CaCl₂, pH 7.4. After washing with 2 column volumes of equilibration buffer without CaCl.sub.2, the recombinant human protein C is eluted with 20 mM Tris, pH 7.4.

Activation. The eluted protein is prepared for activation by removal of residual calcium by passing over a metal affinity column (Chelex-100, Bio-Rad) to remove calcium and again bound to an anion exchanger (Fast Flow Q, Pharmacia). Both of these columns are arranged in series and equilibrated in 20 mM Tris, 150 mM NaCl, 5 mM EDTA, pH 7.4. Following loading of the protein, the Chelex-100 column is washed with one column volume of the same buffer before disconnecting it from the series. The anion exchange column is washed with 3 column volumes of equilibration buffer before eluting the protein with 0.4 M NaCl, 20 mM Tris-acetate, pH 6.5. Protein concentrations of recombinant human protein C and recombinant activated protein C solutions are measured by UV 280 nm extinction E0.1%=1.81 or 1.85, respectively.

Recombinant human protein C (r-hPC) is activated by methods well known in the art. Specifically, r-hPC is activated with bovine thrombin as described in Carlson, et al., U.S. Pat. No. 6,159,468. Bovine thrombin is coupled to Activated CH-Sepharose 4B (Pharmacia) in the presence of 50 mM HEPES, pH 7.5 at 40° C. The coupling reaction is done on resin already packed into a column using approximately 5000 units thrombin/mL resin. The thrombin solution is circulated through the column for approximately 3 hours before adding 2-aminoethanol (MEA) to a concentration of 0.6 mL/L of circulating solution. The MEA-containing solution is circulated for an additional 10-12 hours to assure complete blockage of the unreacted amines on the resin. Following blocking, the thrombin-coupled resin is washed with 10 column volumes of 1 M NaCl, 20 mM Tris, pH 6.5 to remove all non-specifically bound protein, and is used in activation reactions after equilibrating in activation buffer.

Purified r-hPC is made 5 mM in EDTA (to chelate any residual calcium) and diluted to a concentration of 2 mg/mL with 20 mM Tris, pH 7.4 or 20 mM Tris-acetate, pH 6.5. This material is passed through a thrombin column equilibrated at 37° C. with 50 mM NaCl and either 20 mM Tris pH 7.4 or 20 mM Tris-acetate pH 6.5. The flow rate is adjusted to allow for approximately 20 min. of contact time between the r-hPC and thrombin resin. The effluent is collected and immediately assayed for amidolytic activity. If the material does not have a specific activity (amidolytic) comparable to an established standard of aPC, it is recycled over the thrombin column to activate the r-hPC to completion. This is followed by 1:1 dilution of the material with 20 mM buffer as above, with a pH of either 7.4 or 6.5 to keep the aPC at lower concentrations while it awaits the next processing step.

Removal of leached thrombin from the aPC material is accomplished by binding the aPC to an anion exchange resin (Fast Flow Q, Pharmacia) equilibrated in activation buffer (either 20 mM Tris, pH 7.4 or 20 mM Tris-acetate, pH 6.5) with 150 mM NaCl. Thrombin does not interact with the anion exchange resin under these conditions, but passes through the column into the sample application effluent. Once the aPC is loaded onto the column, a 2-6 column volume wash with 20 mM equilibration buffer is done before eluting the bound aPC with a step elution using 0.4 M NaCl in either 5 mM Tris-acetate, pH 6.5 or 20 mM Tris, pH 7.4. Higher volume washes of the column facilitate more complete removal of the dodecapeptide. The material eluted from this column is stored either in a frozen solution (−20° C.) or as a lyophilized powder.

Activity Determination. The anticoagulant activity of activated protein C is determined by measuring the prolongation of the clotting time in the activated partial thromboplastin time (APTT) clotting assay. A standard curve is prepared in dilution buffer (1 mg/mL radioimmunoassay grade bovine serum albumin [BSA], 20 mM Tris, pH 7.4, 150 mM NaCl, 0.02% NaN.sub.3) ranging in protein C concentration from 125-1000 ng/mL, while samples are prepared at several dilutions in this concentration range. To each sample cuvette, 50 μL of cold horse plasma and 50 μL of reconstituted activated partial thromboplastin time reagent (APTT Reagent, Sigma) are added and incubated at 37° C. for 5 min. After incubation, 50 μL of the appropriate samples or standards are added to each cuvette. Dilution buffer is used in place of sample or standard to determine basal clotting time. The timer of the fibrometer (CoA Screener Hemostasis Analyzer, American Labor) is started immediately after the addition of 50 μL 37° C. 30 mM CaCl.2 to each sample or standard. Activated protein C concentration in samples are calculated from the linear regression equation of the standard curve.

### Factor VIII

Methods for expressing and purifying factor VIII are disclosed in U.S. Pat. No. 7,459,525. HKB clones that are adapted to grow as serum-free suspension cultures are further weaned of plasma protein supplements. The weaning is done in sterile polycarbonate shake flasks (Coming, Coming, N.Y.) at a cell density of about 0.5×10⁶ cells/ml using plasma derived protein free medium. The plasma protein free (PPF) medium is DME/F12 medium supplemented with pluronic F68 (0.1%), CuSO4 (50 nM), FeSO₄/EDTA (50 μM), and 5 mM Neu5Ac (N-acetylneuraminic acid). Complete medium exchange is done every 48 hours and the shake flasks are re-seeded at 0.5×10⁶ cells/ml.

A fermenter is seeded with a factor VIII expressing clone with the cells at a density of about 3×106 cells/ml. The fermenter is perfused at a rate of 4 volumes per day with the serum-free production medium as described in the preceding paragraph. A final cell density of 2×10⁷ cells/ml is sustained throughout the evaluation period (45 days). During the first 4 weeks of fermentation, factor VIII expressing clone is perfused with the serum free production medium supplemented with Plasmanate® (Human plasma albumin, Talecris, Research Triangle Park, N.C.) HPP fraction and 5 mM Neu5Ac, and is able to sustain high productivity. From day 28 to the end of the fermentation run, the cells are perfused with the same serum free production medium containing and 5 mM Neu5Ac but without Plasmanate® HPP fraction. The cells continue to produce high levels of FVIII in a plasma derived protein-free environment. “Plasma derived protein-free” means that essentially no proteins isolated from plasma is added to the medium.

### Idursulfase

CHOEFI2S-9 cells are inoculated into two 2-layer cell factories (NUNC, 1200 cm²) in Ham's F12, 10% v/v FCS, and 5 mM Neu5Ac (N-acetylneuraminic acid) and antibiotics. Cells are grown to confluency, the medium removed and the cells are then rinsed 3-times with PBS and re-fed with 200 ml of Ham's F12 without FCS but supplemented with 5 mM Neu5Ac, antibiotics and 10 mM-NH4Cl. After 4 days in culture, the medium are collected and replaced with Ham's F12, 10% v/v FCS, PSK, and 5 mM Neu5Ac but without NH₄Cl for 3 days. This cycle is repeated several times. The conditioned serum free Ham's F12 medium supplemented with NH₄Cl and 5 mM Neu5Ac is collected, clarified by filtration (0.2 μM filture; Millipore) and stored at 4° C.

The recombinant Idursulfase (rIDS) is purified from the collected medium by a 3-step column procedure. The medium is dialysed overnight at 4° C. against 30 mM-Tris/HCl, pH 7.0/10% v/v glycerol/0.1 mM-DTE/3 mM-NaN₃ (buffer A) and is applied to a PBE94 column (8 cm×1.5 cm) equilibrated in buffer A (flow-rate 1.0 ml/min) and then washed with 100 ml of buffer A. Bound proteins are diluted with polybuffer 74 that has been diluted 1:18 with water, the pH adjusted to 4.0 with HCl and the solution made 10% v/v in glycerol, 0.1 mM-DTE and 3 mM-NaN₃. The column is further eluted with 100 ml 15 mM-ditheriothreitol/3 mM-NaN₃ (buffer B). The rIDS eluted in buffer B is applied at a flow-rate of 1.0 ml/min to a Blue A agarose column (6 cm×0.7 cm) also equilibrated in buffer B. The rIDS activity from this step is applied in 1.0 ml volumes to an LKB Ultrachrom GTi f.p.l.c. system with a TSK G3000SW Ultrapac column (30 cm×0.8 cm) equilibrated and eluted in buffer B at a flow-rate of 0.5 ml/min and pressure of 150 kPa. Fractions containing rIDS activity are pooled and analysed under denaturing and nondenaturing condition on SDS-PAGE (10% w/v acrylamide) to estimate apparent subunit size.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Varki, A. Glycan-based interactions involving vertebrate sialic-acid-recognizing proteins. Nature 446, 1023-1029 (2007).
2. Bardor, M., Nguyen, D. H., Diaz, S. & Varki, A. Mechanism of uptake and incorporation of the non-human sialic acid N-glycolylneuraminic acid into human cells. J. Biol. Chem. 280, 4228-4237 (2005).
3. Hedlund, M., Padler-Karavani, V., Varki, N. M. & Varki, A. Evidence for a human-specific mechanism for diet and antibody-mediated inflammation in carcinoma progression. Proc. Natl. Acad. Sci. USA 105, 18936-18941 (2008).
4. Pham, T. et al. Evidence for a novel human-specific xeno-auto-antibody response against vascular endothelium. Blood 114, 5225-5235 (2009).
5. Diaz, S. L. et al. Sensitive and specific detection of the non-human sialic Acid N-glycolylneuraminic acid in human tissues and biotherapeutic products. PLoS ONE 4, e4241 (2009).
6. Hedlund, M. et al. N-glycolylneuraminic acid deficiency in mice: implications for human biology and evolution. Mol. Cell. Biol. 27, 4340-4346 (2007).
7. Padler-Karavani, V. et al. Diversity in specificity, abundance, and composition of anti-Neu5Gc antibodies in normal humans: potential implications for disease. Glycobiology 18, 818-830 (2008).
8. Van Hoeyveld, E. & Bossuyt, X. Evaluation of seven commercial ELISA kits compared with the C1q solid-phase binding RIA for detection of circulating immune complexes. Clin Chem 46, 283-285 (2000).
9. Hara et al., Highly sensitive determination of N-acetyl- and N-glycolylneuraminic acids in human serum and urine and rat serum by reversed-phase liquid chromatography with fluorescence detection., J. Chromatography 377:111-119 (1986).
10. Gagneux, P. et al. Proteomic comparison of human and great ape blood plasma reveals conserved glycosylation and differences in thyroid hormone metabolism. Am J Phys Anthropol 115, 99-109 (2001).
11. Debeire, P., Montreuil, J., Moczar, E., van, H. H. & Vliegenthart, J. F. Primary structure of two major glycans of bovine fibrinogen. Eur. J. Biochem. 151, 607-611 (1985).
12. Chung, C. H. et al. Cetuximab-induced anaphylaxis and IgE specific for galactose-alpha-1,3-galactose. N Engl. J. Med. 358, 1109-1117 (2008).
13. Delbaldo, C. et al. Pharmacokinetic profile of cetuximab (Erbitux) alone and in combination with irinotecan in patients with advanced EGFR-positive adenocarcinoma. Eur. J. Cancer 41, 1739-1745 (2005).
14. Saadeh, C. E. & Lee, H. S. Panitumumab: a fully human monoclonal antibody with activity in metastatic colorectal cancer. Ann Pharmacother 41, 606-613 (2007).
15. Tangvoranuntakul, P. et al. Human uptake and incorporation of an immunogenic nonhuman dietary sialic acid. Proc. Natl. Acad. Sci. USA 100, 12045-12050 (2003).
16. Tahara, H. et al. Immunological Property of Antibodies against N-Glycolylneuraminic Acid Epitopes in Cytidine Monophospho-N-Acetylneuraminic Acid Hydroxylase-Deficient Mice. J. Immunol. 184, 3269-3275 (2010).
17. Taylor, R. E. et al. Novel mechanism for the generation of human xeno-auto-antibodies against the non-human sialic acid N-glycolylneuraminic acid. J. Exp. Med. in press, (2010).
18. Axworthy, D. B. et al. Cure of human carcinoma xenografts by a single dose of pretargeted yttrium-90 with negligible toxicity. Proc. Natl. Acad. Sci. USA 97, 1802-1807 (2000).
19. Hokke, C. H. et al. Sialylated carbohydrate chains of recombinant human glycoproteins expressed in Chinese hamster ovary cells contain traces of N-glycolylneuraminic acid. FEBS Lett. 275, 9-14 (1990).
20. Aggarwal, S. What's fueling the biotech engine-2007. Nat. Biotechnol. 26, 1227-1233 (2008).
21. Arnold, J. N., Wormald, M. R., Sim, R. B., Rudd, P. M. & Dwek, R. A. The impact of glycosylation on the biological function and structure of human immunoglobulins. Annu. Rev. Immunol. 25, 21-50 (2007).
22. Durocher, Y. & Butler, M. Expression systems for therapeutic glycoprotein production. Curr. Opin. Biotechnol. 20, 700-707 (2009).
23. Higgins, E. Carbohydrate analysis throughout the development of a protein therapeutic. Glycoconj. J. (2009).
24. Galili, U. Immune response, accommodation, and tolerance to transplantation carbohydrate antigens. Transplantation 78, 1093-1098 (2004).
25. Noguchi, A., Mukuria, C. J., Suzuki, E. & Naiki, M. Failure of human immunoresponse to N-glycolylneuraminic acid epitope contained in recombinant human erythropoietin. Nephron 72, 599-603 (1996).
26. Borys, M. C. et al. Effects of culture conditions on N-glycolylneuraminic acid (Neu5Gc) content of a recombinant fusion protein produced in CHO cells. Biotechnol Bioeng (2009).
27. Muchmore, E. A., Milewski, M., Varki, A. & Diaz, S. Biosynthesis of N-glycolyneuraminic acid. The primary site of hydroxylation of N-acetylneuraminic acid is the cytosolic sugar nucleotide pool. J. Biol. Chem. 264, 20216-20223 (1989).
28. Qian, J. et al. Structural characterization of N-linked oligosaccharides on monoclonal antibody cetuximab by the combination of orthogonal matrix-assisted laser desorption/ionization hybrid quadrupole-quadrupole time-of-flight tandem mass spectrometry and sequential enzymatic digestion. Anal. Biochem. 364, 8-18 (2007).
29. Jahn, E. M. & Schneider, C. K. How to systematically evaluate immunogenicity of therapeutic proteins—regulatory considerations. N Biotechnol 25, 280-286 (2009).
30. Galili, U. et al. Enhancement of antigen presentation of influenza virus hemagglutinin by the natural human anti-Gal antibody. Vaccine 14, 321-328 (1996).
31. Benatuil, L. et al. The influence of natural antibody specificity on antigen immunogenicity. Eur. I Immunol. 35, 2638-2647 (2005).
32. Abdel-Motal, U. M., Wigglesworth, K. & Galili, U. Mechanism for increased immunogenicity of vaccines that form in vivo immune complexes with the natural anti-Gal antibody. Vaccine 27, 3072-3082 (2009).
33. Koren, E. et al. Recommendations on risk-based strategies for detection and characterization of antibodies against biotechnology products. J. Immunol. Methods 333, 1-9 (2008).
34. Shankar, G., Pendley, C. & Stein, K. E. A risk-based bioanalytical strategy for the assessment of antibody immune responses against biological drugs. Nat. Biotechnol. 25, 555-561 (2007).
35. Wilson, J. M. Medicine. A history lesson for stem cells. Science 324, 727-728 (2009).
36. Martin, M. J., Muotri, A., Gage, F. & Varki, A. Human embryonic stem cells express an immunogenic nonhuman sialic acid. Nat. Med. 11, 228-232 (2005).
37. Martin, M. J., Muotri, A., Gage, F. & Varki, A. Response to Cerdan et al.: Complement targeting of nonhuman sialic acid does not mediate cell death of human embryonic stem cells. Nat. Med. 12, 1115 (2006).
38. Campagnari, A. A., Gupta, M. R., Dudas, K. C., Murphy, T. F. & Apicella, M. A. Antigenic diversity of lipooligosaccharides of nontypable Haemophilus influenzae. Infect. Immun. 55, 882-887 (1987).
39. Greiner, L. L. et al. Nontypeable Haemophilus influenzae strain 2019 produces a biofilm containing N-acetylneuraminic acid that may mimic sialylated O-linked glycans. Infect. Immun. 72, 4249-4260 (2004).

1. A composition comprising a recombinant human glycosylated polypeptide or non-human animal glycosylated polypeptide in which Neu5Gc is substantially reduced.

2. The composition of claim 1, wherein said composition is selected from the group consisting of: a monoclonal antibody, Fc-fusion protein, hormone, cytokine, clotting factor, enzyme inhibitor, enzyme and antiserum.

3. The composition of claim 2, wherein said monoclonal antibody is Tocilizumab, Bevacizumab, Alemtuzumab, Trastuzumab, Adalimumab, Rituximab, Golimumab, Ustekinumab, Panitumumab, Omalizumab, Ibritumomab tiuxetan, Tositumomab-I131, Eculizumab, Canakinumab, Gemtuzumab ozogamicin, Ofatumumab, Palivizumab, Natalizumab, Cetuximab, Infliximab, Abciximab, Basiliximab, Daclizumab, Certolizumab pegol, or Ranibizumab.

4. The composition of claim 2, wherein said Fc-fusion protein is Alefacept, Rilonacept, Etanercept, Abatacept, or Romiplostim.

5. The composition of claim 2, wherein said hormone is Follitropin beta, Follitropin alfa, Luteinizing hormone, Osteogenic Protein-1 (BMP-7), Choriogonadotropin alpha, Thyrotropin alfa, Somatropin, keratinocyte growth factor, Calcitonin, or Platelet-derived growth factor (PDGF).

6. The composition of claim 2, wherein said cytokine is Darbepoetin alfa, Interferon beta-1a, Epoetin beta, Epoetin alfa, Interferon beta-1a, Interferon gamma-1b, Interferon alfacon-1, Interferon alfa-2b, interleukin-1 receptor antagonist (IL-1Ra), Pegfilgrastim, Des-Pro Interleukin-11, G-CSF, IL-2/diphtheria toxin fusion protein, Peginterferon alfa-2a, Aldesleukin (IL-2), or Interferon alfa-2a.

7. The composition of claim 2, wherein said clotting factor is Coagulation factor VIII, Coagulation Factor VIIa, Antihemophilic factor, Coagulation Factor IX, Antihemophilic Factor, or Drotrecogin alfa (Activated Protein C).

8. The composition of claim 2, wherein said enzyme is Alteplase, Laronidase, Imiglucerase, agalsidase-β, yaluronidase, Alglucosidase alfa, N-acetylgalactosamine 4-sulfatase, Human DNase, Tenecteplase, Idursulfase, Collagenase, or Rasburicase.

9. The composition of claim 1, wherein said isolated recombinant glycosylated polypeptide has a mol fraction of Neu5Gc of less than 0.01 mol Neu5Gc sialic acid/mol total sialic acid.

10. The composition of claim 1, wherein said isolated recombinant glycosylated polypeptide has a mol fraction of Neu5Gc of less than 0.1 mol Neu5Gc sialic acid/mol total sialic acid.

11. The composition of claim 1, wherein said isolated recombinant glycosylated polypeptide has a mol fraction of Neu5Gc of less than 2 mol Neu5Gc sialic acid/mol total sialic acid.

12. The composition of claim 1, wherein said recombinant glycosylated polypeptide is isolated from a cell line, wherein said cell line is cultured in media supplemented with a Neu5Gc competitor.

13. The composition of claim 12, wherein said cell line is a non-human animal cell line.

14. The composition of claim 13, wherein said non-human animal cell line is selected from the group consisting of: a hybridoma, Chinese Hamster Ovary (CHO) cell, murine myeloma cell, murine C127 cells, and Baby Hamster Kidney (BHK)

15. The composition of claim 12, wherein said cell line is a human cell line.

16. The composition of claim 15, wherein said human cell line is selected from the group consisting of: HT-1080 and HEK293

17. The composition of claim 12, wherein said Neu5Gc competitor is N-acetylneuraminic acid (Neu5Ac) or N-acetylmannosamine (ManNAc).

18. The composition of claim 12, wherein said media is supplemented with at least 100 μM Neu5Gc competitor.

19. The composition of claim 12, wherein said media is supplemented with at least 1 mM Neu5Gc competitor.

20. The composition of claim 12, wherein said media is supplemented with at least 5 mM Neu5Gc competitor.

21. The composition of claim 1, wherein said recombinant glycosylated polypeptide is isolated from a CMAH-defective non-human animal, wherein said CMAH-defective non-human animal is fed a diet supplemented with a Neu5Gc competitor.

22. The composition of claim 21, wherein said recombinant glycosylated polypeptide is isolated from said CMAH-defective non-human animal.

23. The composition of claim 22, wherein said CMAH-defective non-human animal is selected from the group consisting of: a pig, a goat, a sheep, and a cow.

24. The composition of claim 21, wherein said Neu5Gc competitor is N-acetylneuraminic acid (Neu5Ac) or N-acetylmannosamine (ManNAc).

25. The composition of claim 1, wherein said glycosylated polypeptide, has a mol fraction of Neu5Gc of more than 0.001 Neu5Gc sialic acid/mol total sialic acid and less than 0.5 Neu5Gc sialic acid/mol total sialic acid.
